(19) United States
(12) Patent Application Publication
(10) Pub. No.: US 2024/0312606 A1
(43) Pub. Date: Sep. 19, 2024

(54) MACHINE LEARNING DISTRIBUTED HEALTHCARE DECISION SUPPORT SYSTEM

(71) Applicant: Carlaudy Medical Services, LLC, Gainesville, GA (US)

(72) Inventor: Jason Lee Konzelmann, Gainesville, GA (US)

(73) Assignee: Carlaudy Medical Services, LLC, Gainesville, GA (US)

(21) Appl. No.: 18/185,722

(22) Filed: Mar. 17, 2023

(51) Int. Cl. G16H 40/20; G16H 10/60; G16H 50/20

(52) U.S. Cl. CPC G16H 40/20; G16H 10/60; G16H 50/20

(57) ABSTRACT

A system to optimize hospital unit admission is disclosed. The system comprises a transceiver, a memory, and a processor. The transceiver may receive a request to determine patient eligibility for admission to a hospital unit, and patient medical information associated with a patient. The memory may store a training data and a trained machine module. The trained machine module may be trained using the training data. The training data may include a correlation of training medical information and a plurality of protocols. Each protocol may be associated with a disease and may include criteria for admitting the patient in the hospital unit. The processor may be configured to obtain the request, and execute instructions stored in the trained machine module to determine the patient eligibility based on the medical information, and output the patient eligibility to a user device associated with a medical resource.

### TECHNICAL FIELD

The present disclosure relates to a healthcare decision support system, and more particularly, to a machine learning distributed healthcare decision support system that optimizes process of patient admission to a hospital observation unit.

### BACKGROUND

Modern hospitals have established emergency department observation units (EDOUs) to manage patients in hospitals. An EDOU is a specialized unit designed for efficient assessment of patients before a decision may be made to either discharge or admit the patient to the hospital. The EDOU is typically utilized to avoid unnecessary hospital admission, optimize bed capacity, improve emergency department (ED) and hospital throughput, and deliver efficient patient care.

When a patient arrives at an emergency unit of a hospital, ED medical resources may have to decide whether to admit the patient in the hospital or send the patient to home. The ED medical resources may use the EDOU to monitor the patient for ˜15-48 hours before making such a decision. The ED medical resources may include trained clinicians, nurses, emergency physicians, medical officers, and other healthcare professionals.

Demand for such EDOUs is rising in hospitals due to increase in count of patients seeking hospital admission; however, lack of infrastructure and unavailability of medical resources limit the expansion capacity of EDOUs. Therefore, to manage patient inflow to the EDOUs, the EDOUs may use protocol or rule-based driven patient selection criteria so that only eligible patients may be admitted to the observation units. Such protocols enable the medical resources to make patient disposition decisions in a standardized manner.

Conventionally, such disposition decisions are made by the ED medical resources manually. Manual process of determining patient disposition may be prone to human error and/or biasness.

Thus, there exists a need for a system and method that facilitate optimization of patient admission to protocol driven observation units.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts an example data flow of supervised machine learning for optimizing patient admission to a hospital observation unit in accordance with the present disclosure.

FIG. 3 depicts a block diagram of an example observation unit management system in accordance with the present disclosure.

FIG. 4 depicts a flow diagram of an example hospital unit admission optimization method in accordance with the present disclosure.

### DETAILED DESCRIPTION

### Overview

The present disclosure describes a system and method for optimizing hospital unit admission. Specifically, the present disclosure describes a system to optimize patient admission to a hospital observation unit, such that patients who are eligible for admission may be admitted to the hospital observation unit, and remaining patients may be admitted to other hospital units (e.g., intensive care unit or inpatient beds) or discharged to go home. The system may be an Artificial Intelligence based supervised machine learning system that may use training data to generate a trained machine module, which may facilitate the system to provide accurate patient disposition recommendations. The training data may include correlations of training medical information associated with a plurality of patients and a plurality of protocols against defined patient disposition decisions. Each protocol may be associated with a disease, and may include inclusion and exclusion criteria for admission to the hospital observation unit. The system may determine patient eligibility for admission to the hospital observation unit based on the instructions stored in the trained machine module, and may output a recommendation associated with the determined patient eligibility to a user device associated with a hospital medical resource. Responsive to receiving the recommendation on the user device, the hospital medical resource may make a final disposition decision that may include admitting a patient either to the hospital observation unit or to any other hospital unit (or may discharge the patient to go home).

In an exemplary aspect, when the patient arrives at the hospital, the system may receive a request from a user device associated with a hospital management system or the hospital medical resource to determine patient eligibility for hospital observation unit admission. Responsive to receiving the request, the system may obtain patient's medical information including real-time vital signs and historical medical records. The system may then execute the instructions included in the trained machine module to determine the patient eligibility. The system may further select a protocol, from the plurality of protocols, to be assigned to the patient in the hospital observation unit, when the system determines that the patient may be eligible for hospital observation unit admission. The system may output the selected protocol to the user device associated with a hospital medical resource, so that the patient may be monitored or observed according to the selected protocol in the hospital observation unit.

The system may be further configured to “learn” from the final disposition decisions made by the hospital medical resource and refine the training data to further optimize future patient disposition recommendations. Specifically, the system may compare the patient eligibility recommendations provided by the system and the final disposition decisions made by the hospital medical resource. Responsive to comparing, the system may send a feedback to the training data for refinement and update. The system may further update the trained machine module based on the updated training data, so that future patient eligibility or disposition recommendations may be more accurate.

The present disclosure discloses a system and method to optimize hospital observation unit admission, so that the hospital observation unit may not be overcrowded and at the same time eligible patients are not discharged or admitted to other hospital units (e.g., inpatient hospital bed, intensive care unit). The system uses supervised machine learning algorithms that provide accurate and unbiased patient disposition recommendations. Further, the system “learns” over time from the patient disposition recommendations provided by the system and final disposition decisions made by the hospital medical resources, so that future recommendations are more accurate. Furthermore, since the system uses machine learning training data, the system minimizes human error in patient disposition decisions and provide rule-based and accurate patient disposition recommendations.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

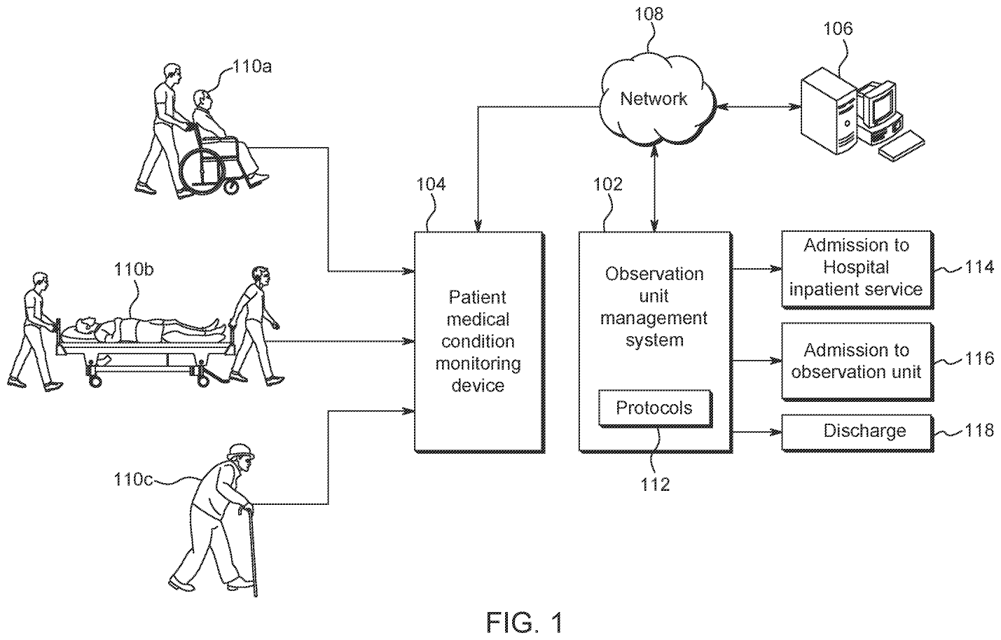

FIG. 1 depicts an example environment 100 in which techniques and structures for providing the systems and methods disclosed herein may be implemented. The environment 100 may include an observation unit management system 102 (or a system 102) connected with a patient medical condition monitoring device 104 (or one or more patient medical condition monitoring devices 104) and a medical record database 106, which may be communicatively connected with each other via one or more networks 108 (or a network 108). In some aspects, the system 102, as described herein, can be implemented in hardware, software (e.g., firmware), or a combination thereof.

The system 102 may be hosted on one or more servers (not shown) and may be an online platform that may facilitate patient admission to one or more units of a hospital (not shown). Specifically, the system 102 may assess medical conditions of one or more patients 110a, 110b, 110c (collectively referred to as patients 110) and determine patient eligibility for admission to an “observation unit” (interchangeably referred as “hospital observation unit”, not shown) of the hospital based on the medical condition, to prevent overcrowding in the observation unit. The system 102 may determine patient eligibility for observation unit admission such that only eligible (or “deserving”) patients may be admitted to the observation unit.

The observation unit may be a specialized hospital unit in which the patient 110 may be admitted for a predefined time duration (e.g., 8 to 48 hours) for observation, and then the patient 110 may either be admitted to a hospital inpatient service or discharged from the hospital observation unit, based on the observation. For example, the patient 110 may be admitted to the hospital inpatient service for further medical assessment and procedure when patient medical condition during the 8 to 48 hour observation time duration in the observation unit does not improve or deteriorates. Alternatively, the patient 110 may be discharged and asked to go home when the patient medical condition during the 8 to 48 hour observation time may improve or may not be critical enough for the patient 110 to be admitted to an inpatient hospital bed

In some aspects, when the patient 110 arrives at the hospital seeking admission to the hospital observation unit (or the hospital inpatient service), the system 102 may assess patient's vital signs and patient's historical medical records and determine whether the patient 110 may be admitted to the hospital observation unit or directly to the hospital inpatient service. The system 102 may further determine whether the 110 may be sent home, e.g., when the patient's vital signs and/or patient's historical medical records do not indicate critical medical condition that may require admission to the hospital observation unit or the hospital inpatient service. Based on the determination, the system 102 may transmit a recommendation indicating an appropriate patient disposition to a device associated with a medical practitioner or hospital staff via the network 108.

The network 108 may be, for example, a communication infrastructure in which the connected devices discussed in various embodiments of this disclosure may communicate. The network 108 may be and/or include the Internet, a private network, public network or other configuration that operates using any one or more known communication protocols such as, for example, transmission control protocol/Internet protocol (TCP/IP), Bluetooth®, BLE®, Wi-Fi based on the Institute of Electrical and Electronics Engineers (IEEE) standard 802.11, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few examples.

The patient medical condition monitoring device 104 may be configured to monitor/detect patient medical condition or vital signs in real-time. In some aspects, the patient medical condition monitoring device 104 may be one or more biometric devices that may be configured to measure patient vital signs. For example, the patient medical condition monitoring device 104 may detect/measure patient body temperature, pulse rate, respiration rate, blood pressure, height, weight, and/or the like.

The medical record database 106 may be a database that may include patient historical medical records. Specifically, the medical record database 106 may include lab records, historical prescriptions, historical treatments or surgeries, family medical history, allergies, and/or the like associated with each patient 110. Such information may be stored against a unique patient identifier. The unique patient identifier may be patient's contact number or a hospital generated identifier (that may be numeric, alpha-numeric and/or the like.), and may be unique to each patient. The system 102 may be configured to extract or fetch patient information from the medical record database 106 by using the unique patient identifier to determine patient eligibility for observation unit admission.

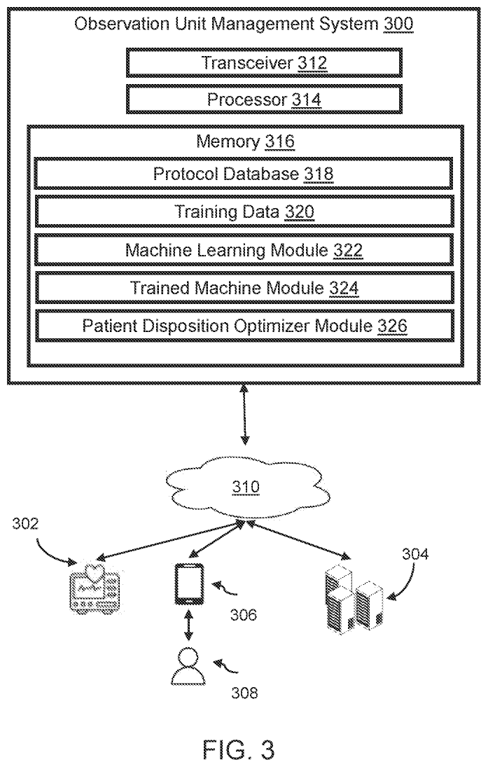

In some aspects, the system 102 may include a plurality of protocols 112 or rule-sets (that may be pre-stored in a system memory, shown as memory 316 in FIG. 3) using which the system 102 may determine patient eligibility for observation unit admission. Specifically, the system 102 may fetch patient vital signs and historical medical records from the patient medical condition monitoring device 104 and the medical record database 106 respectively, and use the plurality of protocols 112 to determine whether to admit the patient 110 in the observation unit or in the hospital inpatient service (or send the patient 110 back home for home care). Each protocol may be associated with a disease, and may include inclusion and exclusion criteria for admitting the patient 110 in the observation unit. For example, the plurality of protocols 112 may include protocols for Cellulitis, Chest Pain, Syncope, and/or the like. In an exemplary aspect, the inclusion criteria associated with a protocol may include patient medical conditions, which when present in the patient 110 may make the patient 110 eligible to be admitted to the observation unit. Similarly, the exclusion criteria may include patient medical conditions, which when present in the patient 110 may make the patient 110 ineligible to be admitted to the observation unit and may be eligible to be admitted to the hospital inpatient service. As an example, the inclusion criteria associated with Cellulitis protocol may include body temperature being less than 38 degrees Celsius, White Blood Count (WBC) being in a range of 4,000 and 12,000, infection band range being less than 10%, and/or the like. In this case, the inclusion criteria may indicate that the patient 110 may need to be observed in the observation unit for a predefined time duration (e.g., 15-24 hours) before a decision may be made to shift the patient 110 to the hospital inpatient service or the patient 110 may be sent back home for home care.

As further example, the exclusion criteria associated with the Cellulitis protocol may include septic or toxic patients (having body temperature greater than 38 degrees Celsius, systolic blood pressure (SBP) less than 90, lactate greater than 4 mmol/L), immunocompromised patients (such neutropenia, HIV, transplant patients, patients on immunosuppressants or chemotherapy), high risk infections such as diabetic foot infections, and/or the like. Such patients may be directly admitted to the hospital inpatient service and may not be required to be admitted to the hospital observation unit.

Similarly, the inclusion criteria associated with chest pain protocol may include no acute electrocardiogram (ECG) changes of acute coronary syndromes (ACS), negative initial troponin (<0.10), Gray zone troponin (between 0.10-0.50) known to be chronic and consistent with previous levels, and/or the like. The exclusion criteria for chest pain protocol may include positive troponin (>0.10) in patients without previous troponin elevation, troponin greater than 0.50 in patients with chronic elevated troponin, new ECG changes consistent with ischemia, and/or the like.

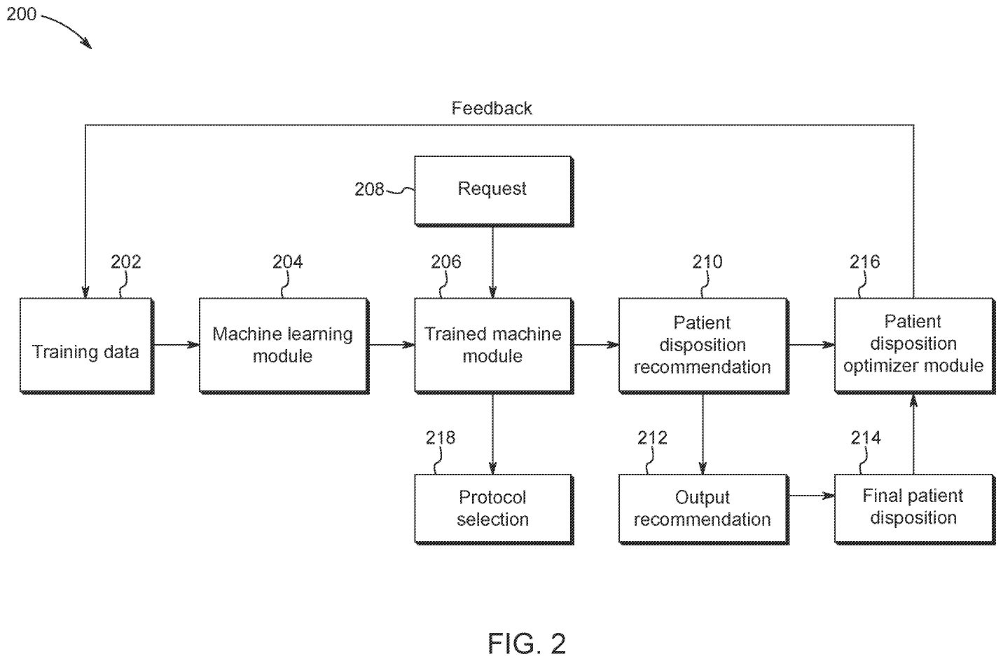

As described above, the system 102 may be configured to facilitate optimization of patient admission to the observation unit, based on the historical medical records, real-time medical condition (obtained from the patient medical condition monitoring device 104), and the plurality of protocols 112. In some aspects, the system 102 may be an Artificial Intelligence (AI) based supervised machine learning system that may use labeled data to output recommendations to admit the patient 110 to the observation unit or to other hospital units. The details of the Artificial Intelligence (AI)-based system are described below later in conjunction with FIG. 2.

In operation, when the patient 110 arrives at the hospital, the ED medical resource may use the patient medical condition monitoring device 104 to detect patient medical condition. Specifically, the patient medical condition monitoring device 104 may measure patient vital signs, and transmit the measured vital signs to the system 102, via the network 108. The system 102 may further receive/obtain historical medical records associated with the patient 110 from the medical record database 106 (using the patient unique identifier), via the network 108. In addition, the system 102 may obtain/fetch the plurality of protocols 112 from the system memory.

Responsive to receiving patient medical information (e.g., information associated with the patient vital signs and historical medical records), the system 102 may execute a machine learning model on the patient medical information based on the plurality of protocols 112 (specifically inclusion and exclusion criteria included in the plurality of protocols 112), to determine patient eligibility for admission to the observation unit (or patient disposition recommendation).

The system 102 may further transmit the patient disposition recommendation to a user device associated with the ED medical resource so that the ED medical resource may use the recommendation for patient disposition. For example, the system 102 may output the patient disposition recommendation as “admit the patient 110 to the hospital inpatient service” (shown as recommendation 114 in FIG. 1), “admit the patient 110 to the observation unit” (shown as recommendation 116 in FIG. 1), or may recommend the patient 110 to go home (shown as recommendation 118 in FIG. 1). In this manner, when the system 102 recommends admitting the patient 110 to the observation unit, the system 102 indicates that the patient 110 may be eligible for observation unit admission. On the other hand, when the system 102 recommends admitting the patient 110 to the hospital inpatient service or discharging the patient 110, the system 102 may indicate that the patient 110 may not be eligible for observation unit admission.

Responsive to a determination that the patient 110 may be eligible for observation unit admission, the system 102 may be additionally configured to select an appropriate protocol from the plurality of protocols 112 and assign the selected protocol to the patient 110 for further procedure in the observation unit, based on the patient vital signs and historical medical records. For example, the system 102 may select and assign Cellulitis protocol for the patient 110, when the medical information associated with the patient 110 (e.g., the patient vital signs and historical medical records) indicates that the patient 110 may be having or suffering from Cellulitis. The system 102 may assign the Cellulitis protocol to the patient 110 so that the patient 110 may be further observed for symptoms of Cellulitis disease in the observation unit.

In further aspects, the system 102 may be configured to determine another patient disposition when the patient 110 may be admitted to the observation unit. In this case, the system 102 may obtain additional medical information (or additional medical dataset) associated with the patient 110 when the patient 110 may be admitted to the observation unit. The additional medical information may be obtained from one or more biometric input devices (e.g., second biometric input devices) that may be disposed in the observation unit. The biometric input devices in the observation unit may be same as or different from the patient medical condition monitoring device 104. In some aspects, the system 102 may obtain the additional medical information associated with the patient 110 at a predefined frequency (e.g., every 15-30 minutes) from the biometric input devices over a predefined time duration of 8 to 48 hours.

Responsive to obtaining the additional medical information, the system 102 may correlate the additional medical information with the selected protocol (e.g., inclusion and exclusion criteria associated with the Cellulitis disease protocol), and determine current patient medical condition based on the correlation. The system 102 may be further configured to determine the other patient disposition based on the current patient medical condition. As an example, if the correlation indicates that the patient body temperature may be more than body temperature range included in the inclusion criterion associated with the Cellulitis disease protocol, the system 102 may provide recommendation (i.e., transmit recommendation to the user device associated with the ED medical resource) to dispose the patient 110 from the observation unit and admit the patient 110 to the hospital inpatient service for further procedure.

FIG. 2 depicts an example data flow 200 of supervised machine learning for optimizing patient admission to a hospital observation unit in accordance with the present disclosure.

A person ordinarily skilled in the art may appreciate that machine learning is an application of Artificial Intelligence (AI) using which systems (e.g., the system 102) may have the ability to automatically learn and improve from experience without being explicitly programmed. Machine learning focuses on use of data and algorithms to imitate the way humans learn. In some aspects, the machine learning algorithms may be created to make classifications and/or predictions. Machine learning based systems may be used for a variety of applications including, but not limited to, speech recognition, email filtering, medical diagnosis, patient disposition decision-making, and/or the like.

Machine learning may be of various types based on data or signals available to the learning system. For example, the machine learning approach may include supervised learning, unsupervised learning, semi-supervised learning, or reinforcement learning. The supervised learning is an approach that may be supervised by a human. In this approach, the machine learning algorithm may use labeled training data and defined variables. In the case of supervised learning, both the input and the output of the algorithm may be specified/defined, and the algorithms may be trained to classify data and/or predict outcomes accurately.

Broadly, the supervised learning may be of two types, “regression” and “classification”. In classification learning, the learning algorithm may help in dividing the dataset into classes based on different parameters. In this case, a computer program may be trained on the training dataset and based on the training, the computer program may categorize input data into different classes. Some known methods used in classification learning include Logistic Regression, K-Nearest Neighbors, Support Vector Machines (SVM), Kernel SVM, Naïve Bayes, Decision Tree Classification, and Random Forest Classification.

In regression learning, the learning algorithm may predict output value that may be of continuous nature or real value. Some known methods used in regression learning include Simple Linear Regression, Multiple Linear Regression, Polynomial Regression, Support Vector Regression, Decision Tree Regression, and Random Forest Regression.

The unsupervised learning is an approach that involves algorithms that may be trained on unlabeled data. An unsupervised learning algorithm may analyze the data by its own and find patterns in input data. Further, semi-supervised learning is a combination of supervised learning and unsupervised learning. A semi-supervised learning algorithm involves labeled training data; however, the semi-supervised learning algorithm may still find patterns in the input data. Reinforcement learning is a multi-step or dynamic process. This model is similar to supervised learning but may not be trained using sample data. This model may learn “as it goes” by using trial and error. A sequence of successful outcomes may be reinforced to develop the best recommendation or policy for a given problem in reinforcement learning.

In an exemplary aspect, the system 102 may use a supervised machine learning module 204 for optimizing patient admission to the hospital observation unit. The supervised machine learning module 204 may be trained using a training data 202 (as labeled data) to generate a trained machine module 206 (e.g., distributed models). Specifically, the supervised machine learning module 204 may generate the trained machine module 206 to optimize patient admission to the hospital observation unit. For example, the supervised machine learning module 204 may generate the trained machine module 206 to reduce unnecessary patient admission to the observation unit, admit serious/critical patients directly to the hospital intensive care unit or hospital inpatient service, and/or the like.

The training data 202 may include correlations of training medical information and the plurality of protocols 112 against defined patient dispositions (e.g., patient admission to the hospital inpatient service or the observation unit, or patient discharge to home). The training medical information may be medical information of a plurality of patients (i.e., not specific to a patient), and may include vital sign ranges, medical history etc. For example, the training data 202 may include correlation indicating that a patient undergoing chemotherapy and having body temperature greater than 38 degrees Celsius, systolic blood pressure (SBP) less than 90, lactate greater than 4 mmol/L, may be admitted to the hospital intensive care unit or hospital inpatient service (as this patient may be critical). Similarly, the training data 202 may include correlation indicating that a patient having body temperature less than 38 degrees Celsius, WBC in the range of 4,000 and 12,000, infection band range less than 10% may be to be admitted to the observation unit (to observe the patient before admitting the patient to the hospital inpatient service or discharging the patient). The training data 202 may be pre-stored in a system memory (such as the memory 316).

The trained machine module 206 may be configured to receive a request 208 when a patient (e.g., the patient 110) arrives at the hospital. In some aspects, the trained machine module 206 may receive the request 208 from a user device (shown as user device 306 in FIG. 3) associated with the ED medical resource. Responsive to receiving the request 208, the trained machine module 206 may be configured to determine and output a patient disposition recommendation 210. The patient disposition recommendation 210 may include recommendation to either admit the patient 110 in the hospital inpatient service or the observation unit, or recommendation to discharge the patient 110 (e.g., ask the patient 110 to go home). The request 208 may include patient identifier associated with the patient 110. In addition, the request 208 may include medical information (real-time vital signs and historical medical records) associated with the patient 110 obtained from the patient medical condition monitoring device 104 and the medical record database 106.

The trained machine module 206 may be configured to determine and output the patient disposition recommendation 210 based on the medical information included in the request 208. For example, the trained machine module 206 may be trained to recommend patient disposition as “admit to the hospital inpatient service” when the medical information indicates that the patient 110 may be an immunocompromised patient. Thus, when the request 208 indicates that the incoming patient 110 may be an immunocompromised patient, the trained machine module 206 may output the patient disposition recommendation 210 as “admit the patient to the hospital”. In some aspects, the trained machine module 206 may be additionally configured to calculate and output probability distribution of one or more patient disposition recommendations based on the medical information. For example, the disposition may have 75% probability of admitting the patient 110 in the hospital inpatient service when the patient may be immunocompromised patient, 20% probability of admitting the patient 110 in the observation unit, 5% probability of discharging the patient (e.g., not to admit the patient 110 in the observation unit or the hospital inpatient service). In this case, the trained machine module 206 may determine the disposition recommendation having the highest probability as the patient disposition recommendation 210.

In some aspects, the trained machine module 206 may be configured to output (shown as 212 in FIG. 2) the patient disposition recommendation 210 to the user device associated with ED medical resources. The ED medical resources may include, but are not limited to, trained clinicians, nurses, emergency physicians, medical officers, and/or other healthcare professionals. Responsive to receiving the patient disposition recommendation 210 on the user device, the ED medical resource may view the patient disposition recommendation 210 and may use the recommendation to generate a final patient disposition 214.

The data flow 200 may further include procedure for optimizing future patient disposition recommendations. In some aspects, the patient disposition recommendation 210 may be fed to a patient disposition optimizer module 216 for improving the training data 202 to output accurate future patient disposition recommendations. Specifically, the patient disposition optimizer module 216 may receive the patient disposition recommendation 210 along with the final patient disposition 214. The patient disposition optimizer module 216 may compare the final patient disposition 214 (made by the ED medical resource) with the patient disposition recommendation 210 (generated by the trained machine module 206). Based on the comparison, the patient disposition optimizer module 216 may determine whether the patient disposition recommendation 210 matches with the final patient disposition 214. The patient disposition optimizer module 216 may transmit feedback to the training data 202 to update or improve the training data 202 based on the comparison. In some aspects, the updated training data may include modified correlations of training medical information and the plurality of protocols 112 against defined patient dispositions, which may be modified based on the feedback. The supervised machine learning module 204 may use the updated training data, and generate an updated trained machine module. In this manner, the training data 202 may continue to improve as more and more feedback may be provided to the training data by the patient disposition optimizer module 216. The updated trained machine module may further optimize patient admission to the observation unit, and provide refined or more accurate patient disposition recommendations based on the updated training data.

In some aspects, the trained machine module 206 (or any other module) may be further configured to select a protocol from the plurality of protocols 112 (shown as protocol selection 218) that may be applicable to the patient 110 when the patient 110 may be admitted to the observation unit. The trained machine module 206 may select the protocol when the trained machine module 206 may output the patient disposition recommendation 210 as “admit the patient to the observation unit”. In some aspects, the trained machine module 206 may select the protocol based on the medical information associated with the patient 110. The selected protocol may then be assigned to the patient 110 so that the patient 110 may be observed in the observation unit according to the assigned protocol.

For example, the trained machine module 206 may select “chest pain” protocol for the patient 110 (to be admitted to the observation unit) when the medical condition associated with the patient 110 may be similar to the medical conditions included in the inclusion criteria of the chest pain protocol. In this case, when the patient 110 may be admitted to the observation unit, the patient 110 may be observed (and/or treated) for chest pain related symptoms.

In further aspects, the trained machine module 206 may output the selected protocol to the user device in addition to the patient disposition recommendation 210. In some aspects, the trained machine module 206 may output the selected protocol and the patient disposition recommendation 210 simultaneously or sequentially.

FIG. 3 depicts a block diagram of an example observation unit management system 300 (or system 300) in accordance with the present disclosure. The system 300 may be same as the observation unit management system 102 described above. In some aspects, the system 300, as described herein, can be implemented in hardware, software (e.g., firmware), or a combination thereof.

The system 300 may be connected with a patient medical condition monitoring device 302 (same as the patient medical condition monitoring device 104), a historical medical records database 304 (same as the medical record database 106), and a user device 306 associated with an ED medical resource 308, via a network 310. The network 310 may be same as the network 108.

The system 300 may include one or more components or units including, but not limited to, a transceiver 312, a processor 314, and a memory 316. In some aspects, the memory 316 may store programs in code and/or store data for performing various system operations in accordance with the present disclosure. Specifically, the processor 314 may be configured and/or programmed to execute computer-executable instructions stored in the memory 316 for performing various system functions in accordance with the disclosure. Consequently, the memory 316 may be used for storing code and/or data code and/or data for performing operations in accordance with the present disclosure.

In one or more aspects, the processor 314 may be disposed in communication with one or more memory devices (e.g., the memory 316 and/or one or more external databases (not shown in FIG. 3)). The memory 316 can include any one or a combination of volatile memory elements (e.g., dynamic random-access memory (DRAM), synchronous dynamic random access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.).

The memory 316 may be one example of a non-transitory computer-readable medium and may be used to store programs in code and/or to store data for performing various operations in accordance with the present disclosure. The instructions in the memory 316 can include one or more separate programs, each of which can include an ordered listing of computer-executable instructions for implementing logical functions.

In some aspects, the memory 316 may include a plurality of modules and databases including, but not limited to, a protocol database 318, training data 320, a machine learning module 322, a trained machine module 324, and a patient disposition optimizer module 326. The protocol database 318 may store information (e.g., inclusion and exclusion criteria) associated with the plurality of protocols 112. The training data 320 may be same as the training data 202, the machine learning module 322 may be same as the machine learning module 204, the trained machine module 324 may be same as the trained machine module 206, and the patient disposition optimizer module 326 may be same as the patient disposition optimizer module 216. The machine learning module 322, the trained machine module 324, and the patient disposition optimizer module 326, as described herein, may be stored in the form of computer-executable instructions, and the processor 314 may be configured and/or programmed to execute the stored computer-executable instructions for performing system functions in accordance with the present disclosure.

The transceiver 312 may be configured to communicate with internal and external devices (e.g., via the network 310). For example, the transceiver 312 may receive a request (e.g., request 208) from the user device 306 or a user device (not shown) associated with hospital information/management system to determine patient eligibility for admission to the observation unit (or any other hospital unit) or determine patient disposition, as described above. The transceiver 312 may be further configured to transmit the request to the processor 314 as and when the transceiver 312 receives the request. The transceiver 312 may further receive/obtain medical dataset (or the patient medical information) associated with the patient 110. As described above, the patient medical information may include real-time patient vital signs obtained from the patient medical condition monitoring device 302, and historical medical records obtained from the historical medical record database 304.

The processor 314 may be configured to obtain the request and the medical information from the transceiver 312. Responsive to obtaining the request, the processor 314 may be configured to fetch the trained machine module 324 from the memory 316. As discussed above, the trained machine module 324 may be trained by using the training data 320, and the training data may include correlations of training medical information and the plurality of protocols 112 against defined patient dispositions. Each protocol may be associated with a disease and may include criteria for admitting the patient in the hospital observation unit. The processor 314 may be further configured to use the instructions stored in the trained machine module 324 and determine the patient eligibility based on the patient medical information. The processor 314 may further output the patient eligibility to the user device 306, via the transceiver 312.

In some aspects, the processor 314 may use the instructions stored in the machine learning module 322 to obtain the training data 320 from the memory 316 and generate the trained machine module 324 using the training data 320.

In further aspects, the processor 314 may be configured to use the instructions stored in the trained machine module 324 to fetch the plurality of protocols 112 from the protocol database 318 and select a protocol from the plurality of protocols 112 based on the patient medical information. In some aspects, the processor 314 may select the protocol responsive to a determination that the patient 110 may be eligible to be admitted to the hospital observation unit. The processor 314 may output the selected protocol to the user device 306, via the transceiver 312.

In further aspects, the processor 314 may be configured to use the instructions stored in the patient disposition optimizer module 326 to obtain a final disposition made by the ED medical resource 308 based on the outputted patient eligibility. The processor 314 may then compare the final disposition and the outputted patient eligibility and output a feedback to the training data 320 based on the comparison to update the training data 320, as described above.

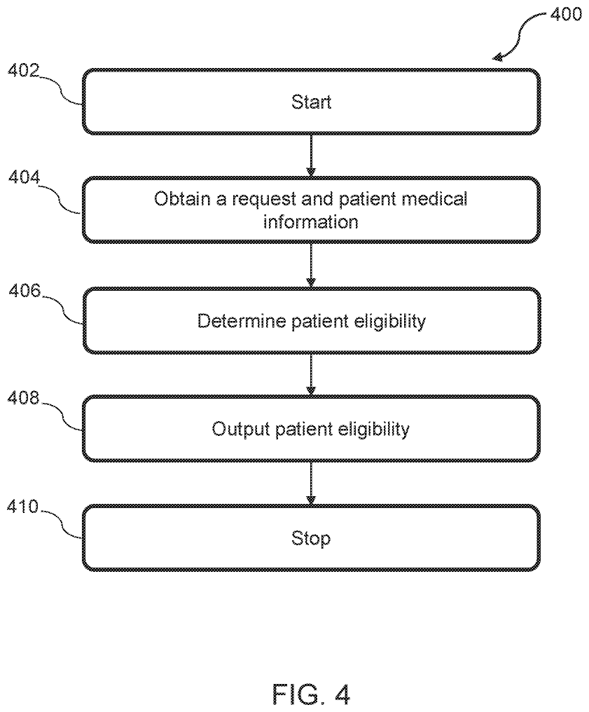

FIG. 4 depicts a flow diagram of an example hospital unit admission optimization method 400 in accordance with the present disclosure. FIG. 4 may be described with continued reference to prior figures, including FIGS. 1-3. The following process is exemplary and not confined to the steps described hereafter. Moreover, alternative embodiments may include more or less steps that are shown or described herein and may include these steps in a different order than the order described in the following example embodiments.

Referring to FIG. 4, at step 402, the method 400 may commence. At step 404, the method 400 may include obtaining, by the processor 314, a request and patient medical information associated with the patient 110. In some aspects, the processor 314 may obtain the request and the patient medical information from the transceiver 312 when the patient 110 arrives at the hospital, as described above. The patient medical information may include real-time patient medical condition (e.g., vital signs) and historical medical records associated with the patient 110.

At step 406, the method 400 may include determining, by the processor 314, patient eligibility to be admitted to the hospital observation unit based on the patient medical information. Specifically, the processor 314 may use the instructions stored in the trained machine module 324 to determine the patient eligibility. For example, the processor 314 may determine whether the patient 110 may be admitted to the hospital observation unit, or the patient 110 may be admitted in the hospital inpatient service (as the patient condition may be serious) or patient 110 may be sent back home (as the patient may get healed by home care).

At step 408, the method 400 may include outputting, by the processor 314, the information associated with the patient eligibility (as recommendation) to user device 306 associated with the ED medical resource 308. The ED medical resource 308 may view the recommendation and may decide the final patient disposition based on the recommendation, as described above.

At step 410, the method 400 may stop.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A system to optimize hospital unit admission, the system comprising:
a transceiver configured to:
receive a request to determine a patient eligibility for admission to a hospital unit, and patient medical information associated with a patient;
a memory configured to store a training data and a trained machine module, wherein:
the trained machine module is trained using the training data,
the training data comprises a correlation of training medical information and a plurality of protocols against defined patient disposition decisions, and
each protocol is associated with a disease and comprises criteria for admitting the patient in the hospital unit; and
a processor communicatively coupled to the transceiver and the memory, wherein the processor is configured to obtain the request and the patient medical information from transceiver and execute instructions stored in the trained machine module to:
determine the patient eligibility based on the patient medical information, responsive to obtaining the request; and
output the patient eligibility to a user device associated with a medical resource.

2. The system of claim 1, wherein the transceiver is configured to receive the patient medical information from one or more first biometric input devices and/or a medical record database.

3. The system of claim 2, wherein the medical record database comprises historical medical condition associated with the patient.

4. The system of claim 1, wherein the hospital unit is a protocol-driven observation unit in a hospital.

5. The system of claim 1, wherein the processor is further configured to:
obtain the training data from the memory; and
generate, via a machine learning module stored in the memory, the trained machine module based on the training data.

6. The system of claim 1, wherein each protocol comprises an inclusion criterion and an exclusion criterion for admitting the patient in the hospital unit.

7. The system of claim 6, wherein the processor is further configured to:
select, via the trained machine module, a protocol from the plurality of protocols responsive to a determination that the patient is eligible for admission to the hospital unit, wherein the selection is based on the patient medical information; and
output the selected protocol to the user device associated with the medical resource.

8. The system of claim 7, wherein the processor is further configured to:
obtain additional medical information associated with the patient from one or more second biometric input devices when the patient is admitted to the hospital unit;
correlate the additional medical information with the inclusion criterion and the exclusion criterion associated with the selected protocol;
determine current patient medical condition in the hospital unit based on the correlation;
determine a patient disposition based on the current patient medical condition, wherein the patient disposition comprises a recommendation to discharge the patient from the hospital unit and a hospital, or a recommendation to admit the patient in a hospital inpatient service; and
output the patient disposition to the user device associated with the medical resource.

9. The system of claim 1, wherein the processor is further configured to:
obtain, via a patient disposition optimizer module stored in the memory, a final disposition made by the medical resource based on the outputted patient eligibility;
compare, via the patient disposition optimizer module, the final disposition and the outputted patient eligibility; and
output, via the patient disposition optimizer module, a feedback to update training data based on the comparison.

10. A method to optimize hospital unit admission, the method comprising:
obtaining, by a processor, a request to determine a patient eligibility for admission to a hospital unit, and patient medical information associated with a patient from a transceiver;
determining, by the processor via a trained machine module, the patient eligibility based on the patient medical information, responsive to obtaining the request, wherein:
the trained machine module is trained using a training data,
the trained machine module and the training data are stored in a memory,
the training data comprises a correlation of training medical information and a plurality of protocols against defined patient disposition decisions, and
each protocol is associated with a disease and comprises criteria for admitting the patient in the hospital unit; and
outputting, by the processor, the patient eligibility to a user device associated with a medical resource.

11. The method of claim 10, wherein the transceiver receives the patient medical information from one or more first biometric input devices and/or a medical record database.

12. The method of claim 11, wherein the medical record database comprises historical medical condition associated with the patient.

13. The method of claim 10, wherein the hospital unit is a protocol-driven observation unit in a hospital.

14. The method of claim 10 further comprising:
obtaining, by the processor, the training data; and
generating, by the processor via a machine learning module stored in the memory, the trained machine module based on the training data.

15. The method of claim 10, wherein each protocol comprises an inclusion criterion and an exclusion criterion for admitting the patient in the hospital unit.

16. The method of claim 15 further comprising:
selecting, by the processor via the trained machine module, a protocol from the plurality of protocols responsive to a determination that the patient is eligible for admission to the hospital unit, wherein the selection is based on the patient medical information; and
outputting, by the processor, the selected protocol to the user device associated with the medical resource.

17. The method of claim 16 further comprising:
obtaining, by the processor, additional medical information associated with the patient from one or more second biometric input devices when the patient is admitted to the hospital unit;
correlating the additional medical information with the inclusion criterion and the exclusion criterion associated with the selected protocol;
determining current patient medical condition in the hospital unit based on the correlation; and
determining a patient disposition based on the current patient medical condition, wherein the patient disposition comprises a recommendation to discharge the patient from the hospital unit and a hospital, or a recommendation to admit the patient in a hospital inpatient service; and
outputting the patient disposition to the user device associated with the medical resource.

18. A non-transitory computer-readable storage medium in a distributed computing system, the non-transitory computer-readable storage medium having instructions stored thereupon which, when executed by a processor, cause the processor to:
obtain a request to determine a patient eligibility for admission to a hospital unit, and patient medical information associated with a patient from a transceiver;
determine, via a trained machine module, the patient eligibility based on the patient medical information, responsive to obtaining the request, wherein:
the trained machine module is trained using a training data,
the trained machine module and the training data are stored in a memory,
the training data comprises a correlation of training medical information and a plurality of protocols against defined patient disposition decisions, and
each protocol is associated with a disease and comprises criteria for admitting the patient in the hospital unit; and
output the patient eligibility to a user device associated with a medical resource.

19. The non-transitory computer-readable storage medium of claim 18, wherein the transceiver receives the patient medical information from one or more first biometric input devices and/or a medical record database.

20. The non-transitory computer-readable storage medium of claim 19, wherein the medical record database comprises historical medical condition associated with the patient.
